(19) United States
(12) Patent Application Publication
(10) Pub. No.: US 2011/0111065 A1
(43) Pub. Date: May 12, 2011

(54) Low Temperature Ultrasonic Extraction Method for Plants

(76) Inventors: Shu-Hua Chiang, Taipei (TW); Ren-Chuan Ko, Taipei (TW)

(21) Appl. No.: 12/633,882

(22) Filed: Dec. 9, 2009

(30) Foreign Application Priority Data
Nov. 10, 2009 (TW) 098138031

(51) Int. Cl. A61K 36/00; A61K 36/258; A61K 36/537; A61K 36/28; A61P 43/00

(52) U.S. Cl. 424/728; 424/725; 424/746; 424/737

(57) ABSTRACT

A low temperature ultrasonic extraction method for plants includes the steps grinding a plant material to obtain plant pieces; immersing the plant pieces in low temperature water for a predetermined time; processing the plant pieces with ultrasonic in the low temperature water to obtain a low temperature extract liquid; and filtering the low temperature extract liquid to remove plant fibers from the extract liquid.

### BACKGROUND OF THE INVENTION

1. Field of the Invention

The present invention relates to a low temperature ultrasonic extraction method for plants. More particularly, the present invention relates to the low temperature ultrasonic extraction method in low temperature water for enhancing the quality of extract from plants.

2. Description of the Related Art

Currently, several extraction methods (for example, hot or warm water extraction method) for plants or Chinese herbal medicines are used in extracting purified products or enzymes. Some constituents contained in the purified products may be good for health. Hence, purified products of plants and Chinese herbal medicines (for example: Anoectochilus formosanus, Ixeris chinensis, coffee, Bupleurum kaoi, Echinacea, Panax ginseng, Sage, Salvia miltiorrhiza, cloud mushroom (or Coriolus versicolor), Ajuga reptens herbaceous plant, Pollygonum cuspidatum or Antrodia Camphorata) will be great benefits to health.

By way of example, Anoectochilus formosanus contain anti-bacterial materials which can suppress breeding of bacterial cells and perform anti cancer effect or anti-bacterial effect, in particular, on tissues of lung cells. Anoectochilus formosanus also can reduce blood concentration and blood glucose level, and can increase the amount of insulin contained in blood.

U.S. Pat. No. 7,504,117, entitled “Extract of Nelumbinis Semen for the treatment of depression,” discloses an ultrasonic extraction method utilizing alcohol or alcoholic solvents for extracting extracts from Nelumbinis Semen. However, extracts of Nelumbinis Semen must be processed in alcohol or alcoholic solvents which will affect the quality of extracts.

U.S. Pat. No. 7,491,414, entitled “Anti-inflammatory substances extracted from Echinacea,” also discloses an ultrasonic extraction method utilizing alcohol or alcoholic solvents for extracting extracts from Echinacea. However, extracts of Echinacea must be processed in alcohol or alcoholic solvents which will affect the quality of Echinacea plant materials.

Taiwanese patent application publication No. 200740377, entitled “Manufacturing method utilizing hydrolyzed vegetable solutions for anti-hypertensive functional foods,” also discloses an ultrasonic extraction method utilizing alcohol or alcoholic solvents for extracting extracts from Benincasa hispida. However, extracts of Benincasa hispida must be processed in alcohol or alcoholic solvents which will affect the quality of extracts. Each of the above-mentioned Taiwanese and US patent application publications and issued patents is incorporated herein by reference for purposes including, but not limited to, indicating the background of the present invention and illustrating the state of the art.

As has been discussed above, there is a need of improving the conventional hot or warm water extraction method to avoid the high temperature affecting the quality of enzymes extracted from plants. Also, there is a need of improving the conventional ultrasonic extraction method utilizing alcohol or alcoholic solvents which will affect the quality of enzymes and mix with the extracts.

As is described in greater detail below, the present invention intends to provide a low temperature ultrasonic extraction method for plants. Plant pieces are immersed in low temperature water for a predetermined time, and are processed by ultrasonic in the low temperature water to obtain a low temperature extract liquid in such a way as to mitigate and overcome the above problem.

### SUMMARY OF THE INVENTION

The primary objective of this invention is to provide a low temperature ultrasonic extraction method for plants. Plant pieces are immersed in low temperature water for a predetermined time, and are processed by ultrasonic in the low temperature water to obtain a low temperature extract liquid. Accordingly, the low temperature ultrasonic extraction method is successful in enhancing the quality of plant extracts and the efficiency of plant extraction.

The low temperature ultrasonic extraction method for plants in accordance with an aspect of the present invention includes the steps:

grinding a plant material to obtain plant pieces;

immersing the plant pieces in low temperature water for a predetermined time;

processing the plant pieces with ultrasonic in the low temperature water to obtain a low temperature extract liquid; and

filtering the low temperature extract liquid to remove plant fibers from the extract liquid.

In a separate aspect of the present invention, the method further comprising the step of: firstly freeze-drying the plant material to remove water content therefrom.

In a further separate aspect of the present invention, the method further comprising the step of: secondly freeze-drying the extract liquid to remove water content for obtaining plant extract powder.

In a yet further separate aspect of the present invention, the method further comprising the step of: sizing the plant piece to form a predetermined thickness suitable for processing ultrasonic extraction.

In a yet further separate aspect of the present invention, the method further comprising the step of maintaining the temperature of the low temperature extract liquid at a predetermined low temperature while processing ultrasonic extraction.

In a yet further separate aspect of the present invention, the temperature of the low temperature water is lower than 45 degrees Centigrade and higher than 25 degrees Centigrade.

In a yet further separate aspect of the present invention, the plant pieces and the low temperature water are mixed at a ratio of 1:12.

In a yet further separate aspect of the present invention, the plant pieces are processed by ultrasonic frequency of 40 KHz.

In a yet further separate aspect of the present invention, the method is suitable for extracting Anoectochilus formosanus with low temperature.

In a yet further separate aspect of the present invention, the method is suitable for extracting Ixeris chinensis, coffee, Bupleurum kaoi, Echinacea, Panax ginseng, Sage, Salvia miltiorrhiza, cloud mushroom (Coriolus versicolor), Ajuga reptens herbaceous plant, Pollygonum cuspidatum or Antrodia Camphorata with low temperature.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a flow chart of a low temperature ultrasonic extraction method for plants in accordance with a preferred embodiment of the present invention.

FIG. 2 is an image of Anoectochilus formosanus extract powder processed by hot water extraction method in accordance with the prior art.

FIG. 3 is an image of Anoectochilus formosanus extract powder processed by the low temperature ultrasonic extraction method in accordance with the preferred embodiment of the present invention.

### DETAILED DESCRIPTION OF THE INVENTION

It is noted that a low temperature ultrasonic extraction method for plants in accordance with the preferred embodiment of the present invention can be applicable to extracting various plants, organic crops, vegetables or Chinese medicine herbals. It will be understood that any ultrasonic device can be applied in executing the low temperature ultrasonic extraction method of the present invention.

Throughout the specification, the term “low temperature,” as used herein, means that the temperature is lower than 45 degrees Centigrade but higher than 25 degrees Centigrade which is suitable for processing ultrasonic extraction in the low temperature water so as to avoid high temperature damage of purified products or enzymes contained in plants.

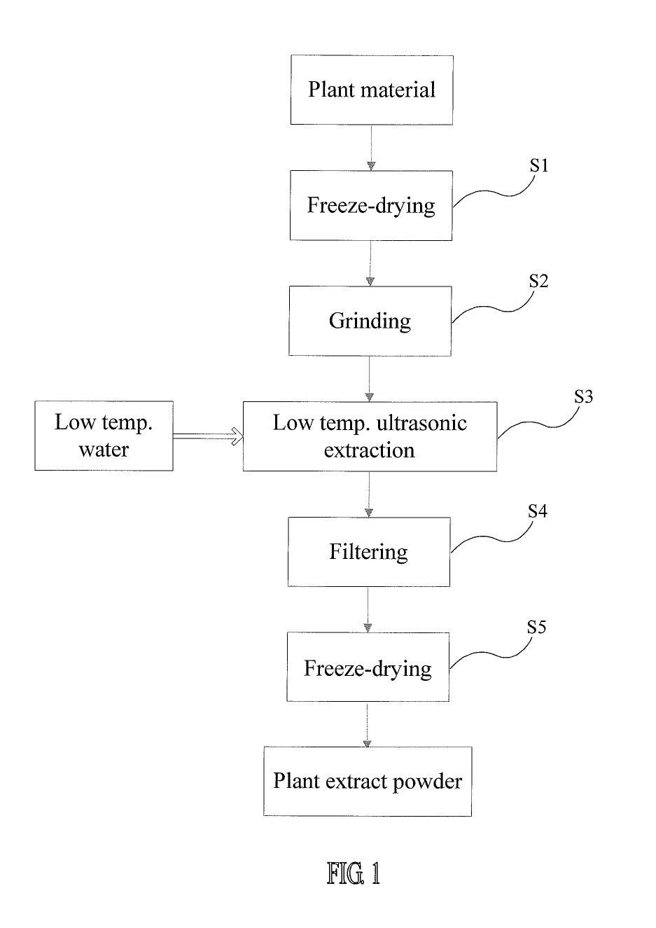

FIG. 1 shows a flow chart of a low temperature ultrasonic extraction method for plants in accordance with a preferred embodiment of the present invention. The low temperature ultrasonic extraction method in accordance with a preferred embodiment of the present invention includes five steps S1, S2, S3, S4 and S5 which can be exchanged or omitted according to the need without departing from the scope of the present invention.

Referring to FIG. 1, in the first step S1 a plant material is processed by a freeze-drying machine for a predetermined time for firstly freeze-drying operation such that water content can be removed from the plant material. In this manner, the dried plant material is prepared.

Still referring to FIG. 1, in the second step S2 the dried plant material is ground in a grinding machine to obtain a predetermined amount of plant pieces. In a preferred embodiment, the plant pieces are sized to form a predetermined thickness suitable for processing ultrasonic extraction.

Still referring to FIG. 1, in the third step S3 the plant pieces are immersed in low temperature water for a predetermined time such that the low temperature water can be fully sucked into the plant pieces. In a preferred embodiment, the temperature of the low temperature water is lower than 45 degrees Centigrade and higher than 25 degrees Centigrade which is suitable for processing ultrasonic extraction in the low temperature water so as to avoid high temperature damage of purified products or enzymes contained in plants.

With continued reference to FIG. 1, the plant pieces and the low temperature water are mixed at a ratio of 1:12. The plant pieces are processed with ultrasonic in the low temperature water to obtain a low temperature extract liquid. Meanwhile, the low temperature water is maintained at a predetermined low temperature by a thermostat during ultrasonic extraction. The temperature of the low temperature extract liquid is further maintained at a predetermined low temperature. The ultrasonic extraction will be terminated if the low temperature extract liquid reaches a predetermined concentration. In a preferred embodiment, the plant pieces are processed by ultrasonic frequency of 40 KHz for 30 minutes which is not limitative of the present invention. In ultrasonic extraction the energy of the ultrasonic will not destroy the purified products or enzymes but will only destroy cell walls of the plant pieces.

Still referring to FIG. 1, in the fourth step S4 a filter is used to filter the low temperature extract liquid for removing plant fibers from the extract liquid.

Still referring to FIG. 1, in the fifth step S5 the low temperature extract liquid is processed by a freeze-drying machine for a predetermined time for secondly freeze-drying operation such that water content can be removed from the low temperature extract liquid to obtain plant extraction powder. Preferably, the plant extraction powder may be instant powder which is not limitative of the present invention.

The low temperature ultrasonic extraction method for plants in accordance with the preferred embodiment of the present invention is suitable for extracting Anoectochilus formosanus, Ixeris chinensis, coffee, Bupleurum kaoi, Echinacea, Panax ginseng, Sage, Salvia miltiorrhiza, cloud mushroom (Coriolus versicolor), Ajuga reptens herbaceous plant, Pollygonum cuspidatum or Antrodia Camphorata with low temperature.

The conventional hot water extraction method and the low temperature ultrasonic extraction method of the present invention are applied to extract Anoectochilus formosanus for producing extract powder. These extract powder extracted from Anoectochilus formosanus are treated by chemical analysis, as will be discussed in greater detail subsequently.

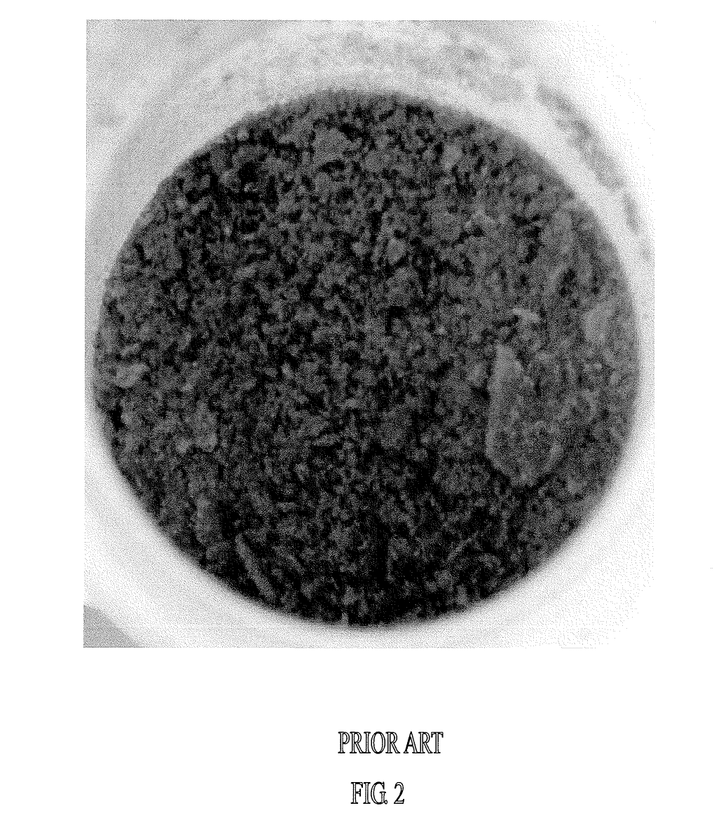

FIG. 2 shows an image of Anoectochilus formosanus extract powder processed by hot water extraction method in accordance with the prior art. In this experiment Anoectochilus formosanus materials are extracted in hot water (100 degrees Centigrade) for 30 minutes to obtain plant extract powder with orange brown color. The plant extract powder has a brightness value (L) of 32.93, a red color value (a) of 15.36 and a yellow color value (b) of 13.04.

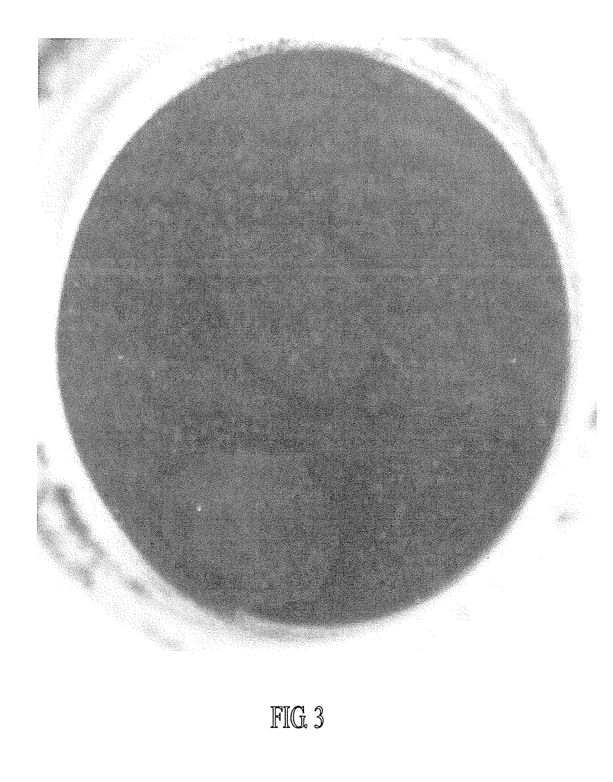

FIG. 3 shows an image of Anoectochilus formosanus extract powder processed by the low temperature ultrasonic extraction method in accordance with the preferred embodiment of the present invention. In this experiment Anoectochilus formosanus materials are extracted by ultrasonic with frequency of 40 KHz in low temperature water (45 degree Centigrade) for 30 minutes to obtain plant extract powder with red color. The plant extract powder has a brightness value (L) of 19.41, a red color value (a) of 13.49 and a yellow color value (b) of 5.49.

Apparently, the low temperature ultrasonic extraction method in accordance with the preferred embodiment of the present invention can retain a greater amount of anthocyanin and natural plant enzymes which may include: proteases, lipase, amylase, celluloses, pineapple enzymes, papaya enzymes, autonomic proteins, and amino nitrogen etc. However, the low temperature ultrasonic extraction method will not result in residual of organic solvents. Conversely, the conventional organic solvent extraction method will result in protein denaturation of the extraction materials and damaging the activation of enzymes.

Referring again to FIGS. 2 and 3, in comparison with the hot water extraction method, the amount of total flavonid contents contained in the plant extraction powder produced by the low temperature ultrasonic extraction method is 2.92 to 3.19 times greater than those produced by the hot water extraction method. Furthermore, the amount of total phenolic contents contained in the plant extraction powder produced by the low temperature ultrasonic extraction method is 1.24 to 1.73 times greater than that produced by the hot water extraction method.

The above-mentioned experimental data and results are provided under preliminary experimental conditions and are not limitative of the present invention. It will be understood that the above-mentioned experimental data and results are only used to demonstrate the present invention. If new experimental data of the present invention are obtained, further analysis will be needed.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. A low temperature ultrasonic extraction method for plants, comprising:
grinding a plant material to obtain plant pieces;
immersing the plant pieces in low temperature water for a predetermined time;
processing the plant pieces with ultrasonic in the low temperature water to obtain a low temperature extract liquid with a predetermined concentration; and
filtering the low temperature extract liquid to remove plant fibers from the extract liquid.

2. The low temperature ultrasonic extraction method as defined in claim 1, further comprising the step of: firstly freeze-drying the plant material to remove water content therefrom.

3. The low temperature ultrasonic extraction method as defined in claim 1, further comprising the step of: secondly freeze-drying the extract liquid to remove water content for obtaining plant extract powder.

4. The low temperature ultrasonic extraction method as defined in claim 1, further comprising the step of: sizing the plant piece to form a predetermined thickness suitable for processing ultrasonic extraction.

5. The low temperature ultrasonic extraction method as defined in claim 1, further comprising the step of: maintaining the temperature of the low temperature extract liquid at a predetermined low temperature while processing ultrasonic extraction.

6. The low temperature ultrasonic extraction method as defined in claim 1, wherein the temperature of the low temperature water is lower than 45 degrees Centigrade and higher than 25 degrees Centigrade.

7. The low temperature ultrasonic extraction method as defined in claim 1, wherein the plant pieces and the low temperature water are mixed at a ratio of 1:12.

8. The low temperature ultrasonic extraction method as defined in claim 1, wherein the plant pieces are processed by ultrasonic frequency of 40 KHz.

9. The low temperature ultrasonic extraction method as defined in claim 1, wherein the low temperature water is maintained at a predetermined low temperature by a thermostat during ultrasonic extraction.

10. The low temperature ultrasonic extraction method as defined in claim 1, wherein the method is suitable for extracting Anoectochilus formosanus with low temperature.

11. The low temperature ultrasonic extraction method as defined in claim 1, wherein the method is suitable for extracting Ixeris chinensis, coffee, Bupleurum kaoi, Echinacea, Panax ginseng, Sage, Salvia miltiorrhiza, cloud mushroom (Coriolus versicolor), Ajuga reptens herbaceous plant, Pollygonum cuspidatum or Antrodia Camphorata with low temperature.
